(19) United States
(12) Patent Application Publication
(10) Pub. No.: US 2005/0196824 A1
(43) Pub. Date: Sep. 8, 2005

(54) Chaperonin and osmolyte protein folding and related screening methods

(76) Inventors: Mark T. Fisher, Shawnee Mission, KS (US); Paul A. Voziyan, Nolensville, TN (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 11/119,845

(22) Filed: May 2, 2005

Related U.S. Application Data
(63) Continuation-in-part of application No. 09/808,774, filed on Mar. 15, 2001, now Pat. No. 6,887,682.
(60) Provisional application No. 60/189,362, filed on Mar. 15, 2000.

(51) Int. Cl. C12Q 1/68; C12Q 1/37; C12P 21/06

(52) U.S. Cl. 435/23; 435/68.1

(57) ABSTRACT

The invention describes an inexpensive in vitro protein folding process for preventing large scale protein misfolding and aggregation, for concentrating aggregation prone chaperonin-protein folding intermediates in a stable non-aggregating form, and for rapidly screening these stable concentrates for the best folding solution conditions. The process comprises: (1) the formation of a chaperone-substrate complex and (2) the release of the substrate using a broad array of folding solutions containing different osmolyte ions, detergents, gradients of ionic strength and pH or other commonly used folding additives. Specifically, when the chaperonin/osmolyte protein process was applied to identify and optimize GSΔ468 bacterial glutamine synthetase mutant refolding conditions that otherwise cannot be folded in vitro by commonly used techniques, 67% of the enzymatic activity was recovered.

### CROSS-REFERENCE TO RELATED APPLICATIONS

This application is continuation-in-part application of U.S. patent application Ser. No. 09/808,774, which was filed on Mar. 15, 2001, now U.S. Pat. No. 6,887,682, which incorporates by reference and claims the benefits and priorities of U.S. Provisional Patent Application No. 60/189,362 filed Mar. 15, 2000.

### STATEMENT REGARDING FEDERALLY SPONSORED RESEARCH OR DEVELOPMENT

Not applicable.

### FIELD OF THE INVENTION

This invention relates to a method of in vitro protein folding. More particularly, the method employs both chaperonins and osmolytes to optimize protein folding as well as to aid in the screening for optimal folding solution conditions.

### BACKGROUND OF THE INVENTION

Efficient refolding of proteins in vitro is an important problem in protein structural analysis and biotechnological manufacturing of pharmaceutical products. Because of their inherent ability to rapidly overexpress proteins to high yields, bacterial systems are the organisms of choice for protein mass production. Unfortunately, overexpression of foreign and, especially, mutant proteins often leads to the development of large intracellular aggregates or inclusion bodies (Rudolph, R and Lilie, H. (1996) FASEB J. 10, 49-56; Guise, A. D., West, S. M., and Chaudhuri, J. B. (1996) Mol. Biotechnol. 6, 53-64, the disclosures of which are incorporated herein by reference). In some cases, the proper intracellular folding of the overexpressed proteins can be enhanced by lowering the cell growth temperature, co-expressing molecular chaperones, or introducing low molecular weight additives (Kujau, M. J., Hoischen, C., Riesenberg, D., and Gumpert, J. (1998) Appl. Microbiol. Biotechnol. 49, 51-58; Tate, C. G., Whiteley, E., and Betenbaugh, M. J. (1999) J. Biol-Chem. 274, 17551-17558; Minning, S., Schmidt-Dannert, C., Schmid, R. D. (1998) J. Biotechnol. 66, 147-156, the disclosures of which are incorporated herein by reference). More often, however, investigators are forced to rely on in vitro folding methods to denature (also known as “deactivate”) and then refold (also known as “reactivate”) aggregated proteins. A number of in vitro approaches have been developed to minimize protein aggregation and enhance proper refolding. Among those are: (1) the addition of osmolytes and denaturants to refolding buffer (Tate, C. G., Whiteley, E., and Betenbaugh, M. J. (1999) J. Biol-Chem. 274, 17551-17558; Plaza-del-Pino, I. M. and Sanchez-Ruiz, J. M. (1995) Biochemistry 34, 8621-8630, Frye, K. J. and Royer, C. A. (1997) Protein. Sci. 6: 789-793, the disclosures of which are incorporated herein by reference); (2) the use of the combinations of different molecular chaperones (Thomas, J. G., Ayling, A., and Baneyx, F. (1997) Appl. Biochem. Biotechnol. 66, 197-238; Buchberger, A., Schroder, H., Hesterkamp, T., Schonfeld, H. J., and Bukau, B. (1996) J. Mol. Biol. 261, 328-233; Veinger, L., Diarnant, S., Buchner, J., and Goloubinoff, P. (1998) J. Biol. Chem. 273, 11032-11037, the disclosures of which are incorporated herein by reference); (3) immobilization of folding proteins to matrices and matrix-bound chaperonins (Stempfer, G., Holl-Neugebauer, B., and Rudolph, R. (1996) Nat. Biotechnol. 14, 329-334; Altamirano, M. M., Golbik, R., Zahn, R., Buckle, A. M., and Fersht, A. R. (1997) Proc. Natl. Acad. Sci. USA 94, 3576-3578; Preston, N. S., Baker, D. J., Bottomley, S. P., and Gore, M. G. (1999) Biochim. Biophys. Acta 1426, 99-109, the disclosures of which are incorporated herein by reference); and (4) utilization of folding catalysts such as protein disulfide isomerase and peptidyl-prolyl cis-trans isomerase (Altamirano, M. M., Garcia, C., Possani, L. D., and Fersht, A. R. (1999) Nat. Biotechnol. 17, 187-191, the disclosure of which is incorporated herein by reference). Unfortunately, because of the diversity of the protein folding mechanisms, there is no universal procedure for protein folding and folding conditions have to be optimized for each specific protein of interest. Therefore, there is always a need for new and more versatile folding techniques. This invention involves a novel protein folding procedure that combines the use of the GroE chaperonins and cellular osmolytes.

Because of its ability to bind many different protein folding intermediates, it was thought that the bacterial GroE chaperonin system could provide a general method to refold misfolded proteins. Chaperonin GroEL is a tetradecamer of identical 57 kDa subunits that possesses two large hydrophobic sites capable of binding to transient hydrophobic protein folding intermediates. The hydrophobic binding site undergoes the multiple cycles of exposure and burial driven by the ATP binding and hydrolysis and the co-chaperonin GroES binding and dissociation. Accordingly, the protein folding intermediates can undergo multiple rounds of binding to and release from the GroEL until they achieve the correctly folded state (for review, see Fenton, W. A. and Horwich, A. L. (1997) Protein Sci. 6, 743-760, the disclosure of which is incorporated herein by reference). Besides simple prevention of non-productive aggregation, chaperonins may also influence the conformation of the folding intermediates, actively diverting them to a productive folding pathway (Fedorov, A. N. and Baldwin, T. O. (1997) J. Mol. Biol. 268, 712-723; Shtilerman, M., Lorimer, G., and Englander, S. W. (1999) Science 284, 822-825, the disclosures of which are incorporated herein by reference). However, despite the general nature of chaperonin-protein interactions, there are many proteins that, for reasons that are currently unknown, cannot fold correctly from the bacterial chaperonin system.

The addition of osmolytes often results in an observed increase in stability of the native structure for some proteins. The stabilization effect is observed with various osmolytes and small electrolytes such as sucrose, glycerol, trimethylamine N-oxide (TMAO), potassium glutamate, arginine and betaine (Wang, A. and Bolen, D. W. (1997) Biochemistry 36, 9101-9108; De-Sanctis, G., Maranesi, A., Ferri, T., Poscia, A., Ascoli, F., and Santucci, R. (1996) J. Protein. Chem. 15, 599-606; Chen, B. L. and Arakawa, T. (1996) J. Pharm. Sci. 85, 419-426; Zhi, W., Landry, S. J., Gierasch, L. M., and Srere, P. A. (1992) Protein Science 1, 552-529, the disclosures of which are incorporated herein by reference). This effect is based on the exclusion of osmolytes from hydration shells and crevices on protein surface (Timasheff, S. N. (1992) Biochemistry 31, 9857-9864, the disclosure of which is incorporated herein by reference) or decreased solvation (Parsegian, V. A., Rand, R. P., and Rau. D. (1995). Methods. Enzymol. 259, 43-94, the disclosure of which is incorporated herein by reference). In a series of quantitative studies, Wang and Bolen have shown that the osmolyte-induced increase in protein stability is due to a preferential burial of the polypeptide backbone rather than the amino acid side chains (Wang, A. and Bolen, D. W. (1997) Biochemistry 36, 9101-9108). Because native protein conformations are stabilized, proper folding reactions are also enhanced in the presence of osmolytes (Frye, K. J. and Royer, C. A. (1997) Protein. Sci. 6: 789-793; Kumar, T. K., Samuel, D., Jayaraman, G., Srimathi, T., and Yu, C. (1998) Biochem. Mol. Biol. Int. 46, 509-517; Baskakov, I. and Bolen, D. W. (1998) J. Biol. Chem. 273: 4831-4834, the disclosures of which are incorporated herein by reference). Osmolytes usually affect protein stability and folding at physiological concentration range of 1-4 M (Yancey, P. H., Clark, M. E., Hand, S. C., Bowlus, R. D., and Somero, G. N. (1982) Science 217, 1214-1222, the disclosure of which is incorporated herein by reference). However, it is apparent that the degree of stabilization depends on both the nature of the osmolyte and the protein substrate (Sola-Penna, M., Ferreira-Pereira, A., Lemos, A. P., and Meyer-Fernandes, J. R. (1997) Eur. J. Biochem. 248, 24-29, the disclosure of which is incorporated herein by reference) and, in some instances, the initial aggregation reaction can actually accelerate in the presence of osmolytes (Voziyan, P. A. and Fisher M. T. (2000) Protein Science, Volume 9, 2405-2412).

Although GroE chaperonins and osmolytes have been used in the folding protocols separately, no studies have taught or suggested the feasibility of combining these two approaches. This invention demonstrates that the combination of chaperonins and osmolytes can provide a considerable advantage in assisting protein folding. Moreover, the method of the present invention can be applied as a more general technique for a rapid identification of the optimal folding solution conditions to achieve maximal yields of correctly folded protein. In particular, the initial off-pathway aggregation is avoided through formation of stable chaperonin-protein substrate complexes under the solution conditions that favor the maximum binding of the substrate to GroEL. These long-lived stable complexes are added to a series of different osmolyte solutions (“folding array”) to identify the most efficient folding conditions for the protein substrate in question.

As a model, this invention examines the in vitro refolding of C-terminal truncation mutant of bacterial glutamine synthetase, GS□468. Unlike native glutamine synthetase (“GS”), this single amino acid truncation product folds to an intermediate that cannot be refolded to an active form by either chaperonins or osmolytes alone. However, the combination of chaperonins and a number of natural osmolytes allowed for the refolding of GSΔ468. Under the optimized conditions, close to 70% of mutant protein refolded to an active form, even at protein concentrations approaching 1 mg/ml.

Therefore, it is an object of this invention to provide an in vitro protein folding process for preventing large-scale protein misfolding and aggregation.

It is a further object to provide a protein folding process that concentrates aggregation prone chaperonin-protein folding intermediates in a stable non-aggregating form.

It is another object of this invention to provide a protein folding process that rapidly screens stable chaperonin-substrate intermediates for the best folding solution conditions.

To accomplish the above and related objects, this invention may be embodied in the detailed description that follows, together with the appended drawings and claims.

### BRIEF DESCRIPTION OF THE DRAWINGS

FIG. 1A and FIG. 1B show the kinetics of spontaneous and chaperonin-dependent renaturation of wild type and mutant GS.

FIGS. 2A and 2B compare the assembly time of wild type GS and GSΔ468 in the presence of chaperoning. The set of arrows in FIG. 2 indicates the GS monomers, dimers, tetramers, and higher multimers produced by time-dependent association of native GS from the chaperonin.

FIG. 3 shows the chaperonin-dependent renaturation of wild type and mutant GS in the presence of glycerol.

FIG. 4 depicts a schematic of a general protein folding screening system that utilizes a combination of chaperonins and osmolytes.

FIG. 5 shows the re-folding of malate dehydrogenase (MDH) using agarose beads upon which a chaperonin has been immobilized.

FIG. 6 shows re-folding of GS on chaperonin beads.

FIG. 7 shows the effectiveness of the GroEL chaperonin at elevated (1M) concentrations of urea.

FIG. 8 shows the aggregation preventive effect of the osmolyte glycerol.

FIG. 9 shows the aggregation preventative effect of the osmolyte urea on rhodanese.

FIG. 10 shows that the osmolyte alone may be sufficient to release the protein from the chaperonin without the addition of ATP.

FIG. 11 shows folding of proteins using GroEL with and without the presence of oxygen.

FIG. 12 illustrates the operation of the chaperonin folding mechanism with an oxidized transient intermediate.

FIG. 13 shows test results for the use of MDH as a folding substrate.

FIG. 14 illustrates immobilization protocols for the chaperonin GroEL, including aminolink or sulfolink chemistries.

FIGS. 15(A) and (B) shows that the same folding success and conditions can be observed with combinations of osmolytes and chaperonins in solution are observed when the double ring GroEL chaperonin is immobilized.

FIG. 16 is a flowchart showing the methodology for the macroscale chaperonin/osmolyte protein folding screening approach to identify optimal osmolyte systems.

FIG. 17 is a flowchart showing the methodology for the macroscale chaperonin/osmolyte protein folding and purification system using a column chromatography approach.

FIG. 18 is a flowchart showing the methodology for the macroscale chaperonin/osmolyte protein folding and purification system using an ultrafiltration separation technique.

### DETAILED DESCRIPTION OF PREFERRED EMBODIMENT

### I. Materials

As used herein, “protein” is defined as a polypeptide or polypeptide chain having a native or “active” form with a known biological function and a denatured form which does hot exhibit the biological function of the native form.

As used herein, “chaperonin” is defined as any protein complex that binds to an unfolded polypeptide to facilitate the folding of said polypeptide to its biologically active state either independently or with the assistance of other elements. This definition specifically includes but is not limited to chaperonin systems from bacteria and bacteriophages, including mesophiles and thermophilic chaperoning. Similarly, as used herein, chaperonin includes but is not limited to chaperonins in any native or modified state, for example, single ring chaperonings glutaldehyde cross-linked chaperonins or other chemically modified chaperoning.

As used herein, “unfolded”, “denatured” and “inactive” are defined interchangeably to mean the characteristic of polypeptides which are no longer biologically active due, at lease in part, to not being in their native shape. As such, the terms include partially folded proteins, chemically unfolded proteins, thermally denatured proteins, pressure unfolded proteins, and oxidatively damaged proteins.

Urea was purchased from ICN Biochemical (Aurora, Ohio.). Trimethylamine N-oxide dehydrate, potassium glutamate, betaine monohydrate, sarcosine hydrochloride, and ATP were from Sigma-Aldrich (St. Louis, Mo.). Glycerol and sucrose were purchased from Fisher Scientific (Pittsburgh, Pa.). All the above chemicals were over 99% pure. The other chemicals were of analytical grade.

Wild type GS was purified from E. coli as previously described (Fisher, M. T. and Stadtman, E. R. (1992) J. Biol. Chem. 267, 1872-1880, the disclosure of which is incorporated herein by reference). A single amino acid C-terminal truncation mutant GSΔ468 was a gift from Dr. R. Stoffel and Dr. Joe Villafranca (Stoffel, R. H., III. (1994) Thesis of Ph.D. Dissertation. The Pennsylvania State University, the disclosure of which is incorporated herein by reference). The E. coli chaperoning, GroEL and GroES were isolated from overexpression E coli strains kindly provided by Drs. Edward Eisenstein and George Lorimer (respectively) and these proteins were purified essentially as described earlier (Fisher, M. T. (1992) Biochemistry 31, 3955-3963; Eisenstein, E., Reddy, P., and Fisher, M. T. (1998). Methods. Enzymol. 290, 119-135; Fisher, M. T. (1994) J. Biol. Chem. 269,13629-13636, the disclosures of which are incorporated herein by reference). The GroEL purification protocol was modified by introducing an additional acetone precipitation step. After the Affi-Gel Blue treatment, GroEL samples were precipitated in 45% (v/v) acetone at room temperature for 5 minutes. The precipitate was centrifuged at 10,000 g for 30 minutes and, after the removal of acetone, re-suspended in 50 mM TrisHCl, 10 mM KCl, 5 mM MgCl2 (pH 7.5). Residual protein aggregates and acetone were removed by a brief centrifugation followed by an extensive dialysis against the above mentioned buffer. The acetone precipitation step significantly improved quality (as measured by silver stained SDS-PAGE gels, tryptophan fluorescence, and second derivative analysis of the UV absorbance spectra) of those GroEL samples with minor impurities that could not be sufficiently purified by Affi-Gel Blue treatment alone. Acetone precipitation did not affect the functional properties of GroEL and can be used as an alternative to the ion-exchange chromatography in methanol for removing minor impurities from GroEL preparations (Todd, M. J. and Lorimer, G. H. (1998) Methods. Enzymol. 290, 136-144, the disclosure of which is incorporated herein by reference).

Molecular chaperones DnaK, DnaJ, and GrpE were purchased from Stress-Gene. Antibodies to E. coli GS were raised in sheep as described by Hohman and Stadtman (Hohman, R. J., Stadtman, E. R. (1978) Biochem. Biophys. Res. Commun. 82, 865-870, the disclosure of which is incorporated herein by reference).

### II. Denaturation and Control Renaturation of GS.

Wild type and mutant GS were denatured in solutions containing 50 mM Tris-HCl (pH 7.5), 5 mM EDTA, 10 mM DTT, and 8 M urea. The denaturation was performed for 4 hours at 0° C. The spontaneous refolding reaction from the denatured protein stock was initiated by a rapid 100-fold dilution of a small concentrated aliquot into either 50 mM Tris-HCl (pH 7.5), 5 mM MgCl2, 50 mM KCl, 0.5 mM EDTA, and 10 mM DTT (buffer A), or into buffer A containing different additives at 37° C., followed by incubation at this temperature. Final GSΔ468 or wild type GS concentration was 0.3 μM.

For the chaperonin-dependent refolding, denatured GS subunits were diluted into buffer A containing either 1 μM GroEL or 1 μM GroEL and 2 μM GroES to form a GroEL-GS complex. After the incubation for 30 minutes at 37° C., either 5 mM ATP alone or ATP and different osmolytes were added and incubation continued for up to 40 hours. In some experiments, GroEL-GS complexes were concentrated using Centricon-30 centrifugation concentrators (Amicon, Inc., Beverly, Mass. ) as described previously (Fisher, M. T. (1993) J. Biol. Chem. 268, 13777-13779, the disclosure of which is incorporated herein by reference), prior to the addition of ATP and/or osmolytes. Centrifugation was performed at 37° C. for 30 minutes. GS activity was determined by the glutamyl transferase assay (Woolfolk, C. A., Shapiro, B., and Stadtman, E. R. (1966) Arch. Biochem. Biophys. 116, 177-192, the disclosure of which is incorporated herein by reference).

### III. Separation and Analysis of GS Renaturation Reaction Products

To characterize the time-dependent changes of the GS species during chaperonin renaturation, nondenaturing gradient gel electrophoresis was used as described before (Fisher, M. T. (1994) J. Biol. Chem. 269,13629-13.636). Briefly, the aliquots of GS renaturation reaction were applied to 8-25% polyacrylamide gradient gel (Pharmacia) at different times after the initiation of refolding. After the rapid (15-20 minutes) separation using the Pharmacia Phast system, the samples were electroblotted to nitrocellulose membrane and analyzed by Western blot using anti-GS antibody and the appropriate secondary antibody linked to alkaline phosphatase (Pierce Chemical Co.).

### IV. Refolding of GSΔ468 from Concentrated Chaperonin Complexes

For the chaperonin-dependent refolding, denatured GSΔ468 was initially diluted into refolding buffer with either 2 μM GroEL alone or 2 μM GroEL and 4 μM GroES to a final GSΔ468 concentration of 0.3 μM. After the formation of GSΔ468-chaperonin complex (10 minutes at 37° C.), samples were concentrated at 37° C. as previously described. Glycerol and ATP were added to respective concentrations of 4 M and 5 mM bringing final GSΔ468 concentration to 7 μM. For spontaneous refolding, the urea-unfolded GSΔ468 was rapidly diluted 100-fold into the refolding buffer (50 mM Tris-HCl, pH 7.5, 0.5 mM EDTA, 50 mM KCl, 5 mM MgCl2) containing 4 M glycerol to a final concentration of 7 μM. Samples were incubated at 37° C. for up to 40 hours and GSΔ468 activity was determined.

### V. Reactivation of Wild and Mutant GS

A. Native Activity and Refolding of Wild Type and Mutant GS.

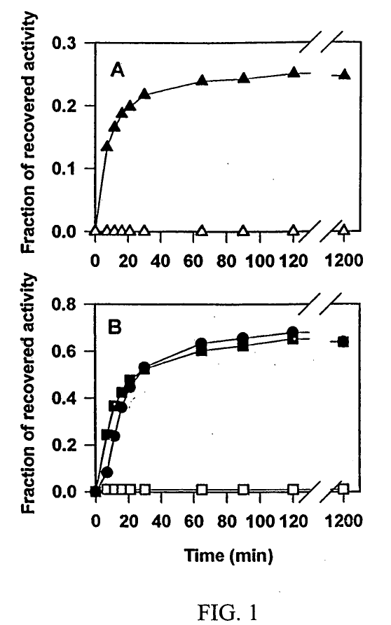

Wild type GS and a single amino acid C-terminal truncation mutant GSΔ468 were produced in bacterial expression system YMC10/pgln6. The assembly of GS into active dodecamer involves swapping of the C-terminal regions of individual subunits and may be affected by truncation. Interestingly, both proteins purified to homogeneity from bacterial lysates were enzymatically active with the specific activity of the mutant GS comprising over 60% of wild type GS activity in a protein concentration range from 0.1 μM to 0.5 μM. Surprisingly, as shown in FIG. 1A, when the purified proteins were denatured in 8 M urea and refolded, the significant recovery of activity was detected only with wild type GS; the urea-denatured truncation mutant could not correctly reassemble and reactivate at all. More importantly, as depicted on FIG. 1B, the GroE chaperonins that enhance the refolding of wild type GS (Fisher, M. T. (1992) Biochemistry 31, 3955-3963), could not reactivate the GSΔ468 truncation mutant.

B. Co-Chaperonin Refolding of Wild and Mutant GS.

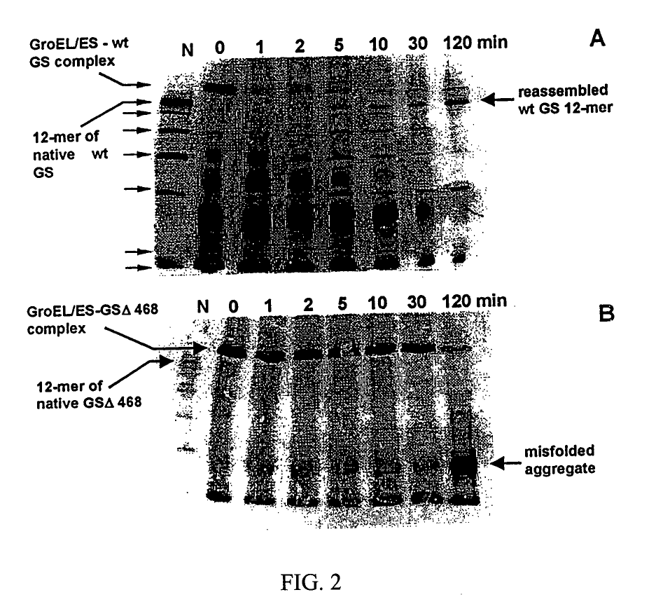

In order to determine why GSΔ468 failed to reactivate with chaperoning, a comparison was made between the time dependent assembly of wild type and mutant GS proteins using non-denaturing gel-electrophoresis and Western blot analysis (Fisher, M. T. (1994) J. Biol. Chem. 269,13629-13636). FIG. 2A shows that upon the addition of GroES and ATP to the GroEL-wild type GS complex, this complex was no longer visible and the assembly of folding monomers into the native dodecamer was largely completed within 2 hours at 37° C. In contrast, FIG. 2B shows that the GSΔ468-chaperonin complex remained visible throughout the time course of the experiment. Furthermore, unlike the wild-type GS, the truncation mutant did not form any native intermediate species after the dissociation from the chaperonin system. Instead, at the end of the time course, non-native aggregates, presumably aberrant dimers and tetramers of the mutant GS have accumulated (FIG. 2B, 120 minutes lane). Thus, GSΔ468 intermediates appear to bind to the chaperonin but are unable to attain an assembly-competent state after their dissociation from the chaperonin complex.

C. Chaperonin-Dependent Refolding of GSΔ468 in the Presence of Molecular Chaperones.

It has been demonstrated that a combination of molecular chaperones such as bacterial DnaK and GroE systems, can augment refolding of proteins that interact with the chaperonins yet fail to fold properly (Buchberger, A., Schroder, H., Hesterkamp, T., Schonfeld, H. J., and Bukau, B. (1996) J. Mol. Biol. 261, 328-233, Petit, M. A., Bedale, W., Osipiuk, J., Lu, C., Rajagopalan, M., McInerney, P., Goodman, M. F., Echols, H. (1994) J. Biol. Chem. 269, 23824-23829, the disclosures of which are incorporated herein by reference). However, the inclusion of the GroE and DnaK/DnaJ/GrpE systems with the GSΔ468 did not result in reactivation of the mutant protein. Change in the folding temperature of this system from 37° C. to 22° C. also failed to refold the truncation mutant.

D. Refolding of GSΔ468 in the Presence of Cellular Osmolytes Only.

Solution additives such as low molecular weight osmolytes have been shown to induce protein folding in vitro, presumably by stabilizing protein native conformation (Wang, A. and Bolen, D. W. (1997) Biochemistry 36, 9101-9108). The present invention examined the effects of several cellular osmolytes on the refolding of GSΔ468. Of all the compounds, only glycerol and, to the lesser extent, sucrose, induced mutant GS refolding. Even so, as shown in Table 1, the recovery of activity under these conditions was very low.

TABLE 1
Refolding of GSΔ468 with GroE chaperonins and osmolytes at 37° C.

| | Activity recovered after 20 hours(fraction of native) | | |
|---|---|---|---|
| | | with | with |
| Osmolyte | Osmolyte alone | GroEL-ATP | GroEL-GroES-ATP |
| 1 M betaine | below assay | 0.13 ± 0.01 | 0.13 ± 0.01 |
| | detection limit | | |
| 1 M sarcosine | << | 0.04 ± 0.01 | 0.20 ± 0.06 |
| 1 M sucrose | 0.05 ± 0.02 | 0.36 ± 0.07 | 0.30 ± 0.07 |
| 0.5 M KGlu | << | 0.09 ± 0.01 | 0.35 ± 0.06 |
| 1 M TMAO | << | 0.22 ± 0.05 | 0.45 ± 0.09 |
| 4 M glycerol | 0.18 ± 0.04 | 0.48 ± 0.08 | 0.47 ± 0.09 |

E. Chaperonin-Dependent Refolding of GSΔ468 in the Presence of Cellular Osmolytes.

However, when osmolytes were added to the chaperonin-GSΔ468 complex, a dramatic synergistic enhancement of protein reactivation was observed. After the formation of GSΔ468-chaperonin complex (10 minutes at 37° C.), respective osmolyte and 5 mM ATP were added. Samples were incubated at 37° C. for 20 hours and GSΔ468 activity was determined as described herein. Final GSΔ468 concentration was 0.3 μM. The data in Table 1 represent the mean±standard deviation of three separate experiments. Not all the tested osmolytes gave the same results. Curiously, the addition of TMAO, potassium glutamate, betaine, and sarcosine worked only with the chaperonins i.e., neither folding enhancer alone produced any effect. This indicates that, in some cases, osmolyte enhanced refolding could only occur from the preexisting chaperonin-GSΔ468 complex.

For some of the osmolytes (TMAO, potassium glutamate, and sarcosine) the GSΔ468 reactivation increased significantly when both GroEL and GroES were present compared to the reactivation with GroEL alone. With glycerol and betaine, however, GroES addition did not improve the yields achieved with GroEL and ATP alone. Since the reactivation yields were optimal with glycerol and protein reactivation did not depend on the presence of co-chaperonin, the GSΔ468 refolding under this solution condition was examined in more detail.

The present invention will be greater explained in the following examples. However, the scope of the invention is not restricted in any way by these examples.

### EXAMPLE 1

### Single Chaperonin plus Osmolyte Folding

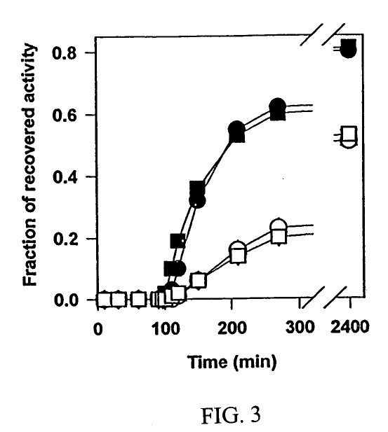

FIG. 3 shows Chaperonin-dependent renaturation of wild type and mutant GS in the presence of glycerol. Urea-denatured GS species were rapidly diluted into refolding buffer at 37° C. with either 1 μM GroEL alone (circles) or lqM GroEL and 2 μM GroES (squares). The activity of GS proteins was followed for 90 min. Upon the addition of 5 mM ATP and 4 M glycerol, the measurements of enzymatic activity of wild type (filled symbols) and mutant (open symbols) GS were continued. Final concentration of GS species was 0.3 μM.

In 4 M glycerol, the kinetics of chaperonin-dependent refolding of GSΔ468 was slower than that of wild type GS; after the incubation for 20 to 40 hours at 37° C. it recovered about 50% of its initial activity. Refolding kinetics of the mutant protein were similar regardless of the presence of GroES, confirming that optimal folding of the mutant could be achieved without the co-chaperonin. This illustrates that solution conditions can be found where GroES is not needed for reactivation, an important consideration for the purification of the refolded protein.

### EXAMPLE 2

### Concentration of Chaperonin-Protein Complexes

This method also works under conditions where larger quantities of folded product are needed. Applicants have previously demonstrated that the GroEL-protein substrate complexes can be routinely concentrated with little loss in recovery of wild type GS and rhodanese (Fisher, M. T. (1993) J. Biol. Chem. 268, 13777-13779; Smith, K. E. and Fisher, M. T. (1995) J. Biol. Chem. 270, 21517-21523, the disclosures of which are incorporated herein by reference). In the present invention, the GSΔ468-GroEL complexes were formed at an optimal substrate-to- chaperonin molar ratio (2:1) and then concentrated about 25-fold. The control experiment showed that only about 1% of the protein was lost in this concentration step. Importantly, very little spontaneous refolding occurred in glycerol solutions at this higher initial concentration of GSΔ468 (Table 2). However, after the chaperonin-GSΔ468 complexes were formed and concentrated, the refolding yields of the truncated GS mutant were as high as 67% of the original activity after 40 hours at 37° C., comparable with refolding yields of wild type GS.

TABLE 2
Refolding of GSΔ468 in 4 M glycerol following concentration
of GroEL-GSΔ468 complexes.

Fraction of recovered activity

| Refolding conditions | after 20 hours | after 40 hours |
|---|---|---|
| Spontaneous | 0.04 | 0.04 |
| GroEL-ATP | 0.64 | 0.67 |

### EXAMPLE 3

### Demonstration that Immoblized GroEL Can Function to Refold Polypeptides

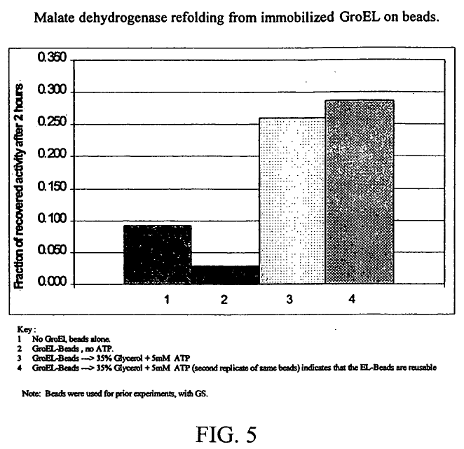

GroEL can be immobilized on inert supports (in this case agarose beads) and can bind unfolded proteins. The immobilized system functions identically to the conditions found in solution (in that addition of osmolytes promises renaturing of the chaperonin complexed proteins). FIG. 5 shows the results of the refolding of MDH using GroEL chaperonin affixed to agarose beads.

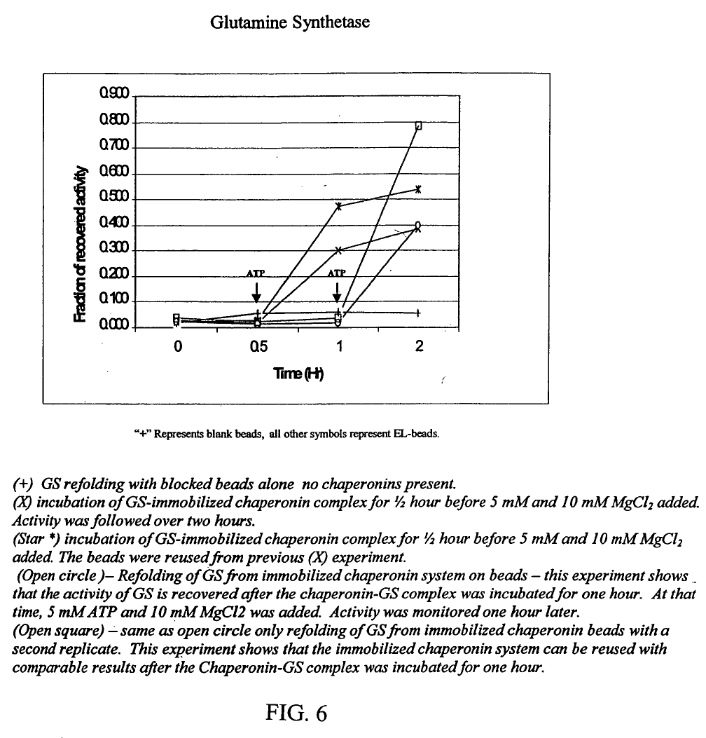

FIG. 6 shows like results for the refolding of GS on GroEL beads. Refolding of GS from immobilized chaperonin system. The immobilized chaperonin can be reused. There is no apparent decline in reactivated activity when the beads are incubated for an extra half hour at 37° C.

### EXAMPLE 4

### Functioning of GroEL at 1M urea

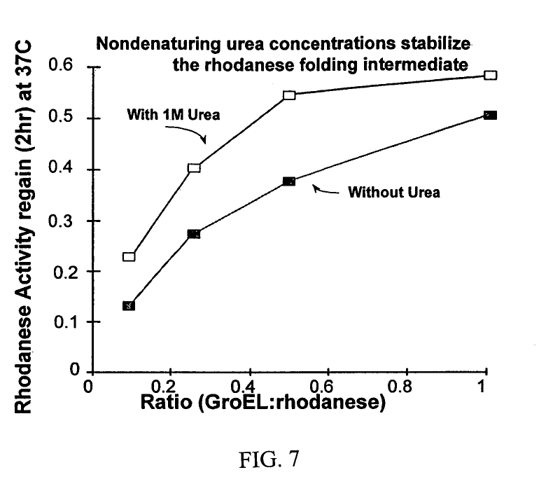

GroEL can function as an effective chaperonin in 1M urea. FIG. 7 shows that even at the 1M urea concentration, GroEL operates to effectively assist with the refolding of the rhodanese. The unexpected synergism of the chaperonin/osmolyte system is again seen in this example.

### EXAMPLE 5

### Prevention of Aggregation by Osmolytes

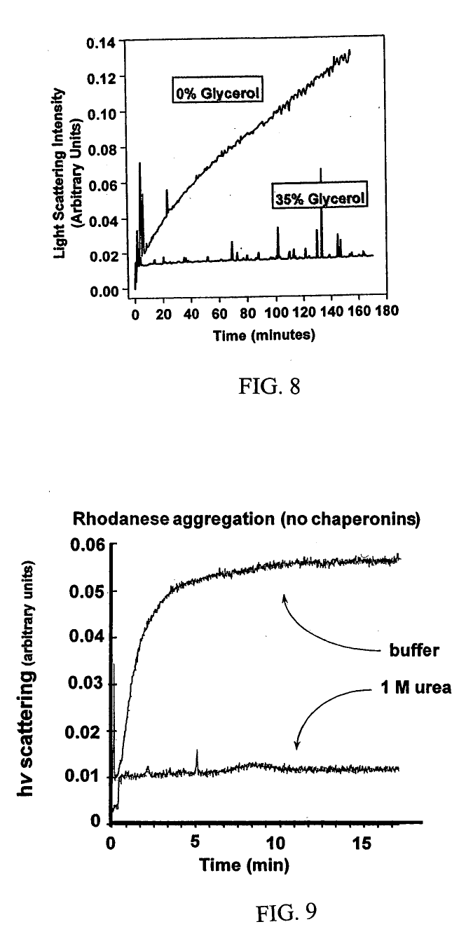

Osmolytes can prevent aggregation. For example, FIG. 8 shows that MDH is substantially prevented from aggregating into unusable forms by the addition of the osmolyte glycerol in a 35% concentration to the solution. Similarly, FIG. 9 shows significant aggregation of rhodanese being avoided by exposure to 1M urea. These examples support the use of iterative (multiple) additions of unfolded polypeptide to increase the yield of chaperonin-protein complexes and to subsequently increase the yield of reactivable protein from the chaperonin. Because these solution conditions prevent large scale aggregation, they increase the capture efficiency of the chaperonin for the soluble partially folded or unfolded protein.

### EXAMPLE 6

### Chaperonin Induced Release of the Protein

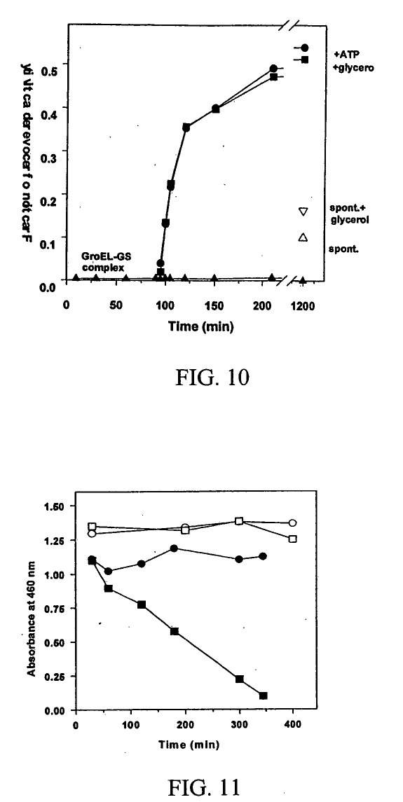

FIG. 10 shows another characteristic of the chaperonin/osmolyte system. It can readily be seen that the release of GS from the GroEL chaperonin was nearly identical for the chaperonin plus osmolyte combination as for the chaperonin plus osmolyte plus ATP combination. As such, the osmolyte alone can induce the release of the folded protein from the chaperonin without the aid of ATP.

### EXAMPLE 7

### Reduction/Oxidation Operation of Chaperonin System (no osmolytes Present)

Chaperonin refolding can be run under anaerobic conditions. FIG. 11 shows GroEL dependent reactivation of rhodanese with and without oxygen (without an osmolyte). Rhodanese (1 μM) was incubated with (▪, □) or without (●, ∘) 10 μM GroEL at 37° C. Data represented by open symbols were obtained under anaerobic conditions as described in Smith K. S., Voziyan P. A. and Fisher M. T., (1998) J. Biol. Chem. 273 28677-28681, incorporated herein by reference.

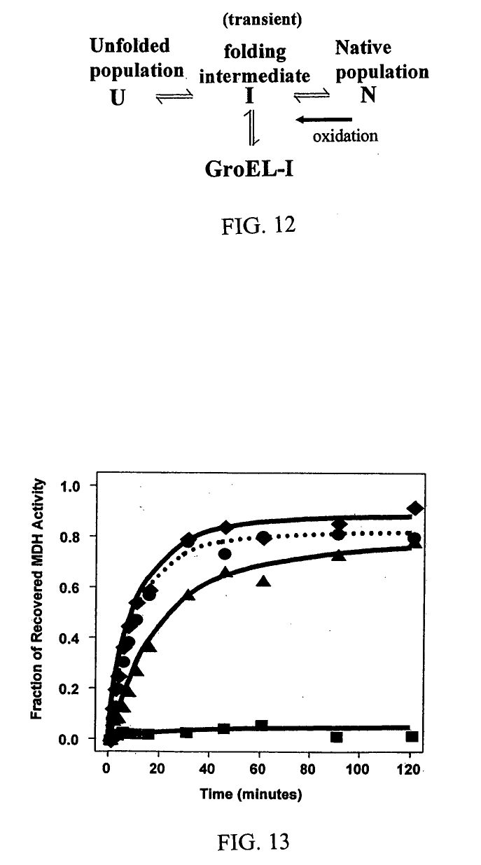

FIG. 12 illustrates the mechanics of the oxidation reaction during the folding operation. As shown, the chaperonin binds a transient oxidized intermediate that is in equilibrium with the native folded population of proteins. Thus, the chaperonin prevents the irreversible oxidation of the folded protein from occurring and the refolding rates from the chaperonin are the same, regardless of the origin (oxidized or non-oxidized) of the intermediate.

For oxygen sensitive folding systems, a number of solution options are available to enhance the success of the chaperonin/osmolyte system. As illustrated in Example 7, the chaperonin/osmolyte system can be used in an inert oxygen free atmosphere (i.e. anaerobic atmospheres) to facilitate protein folding reactivation that is oxygen sensitive. Enhanced folding can also be insured with the osmolyte/chaperonin system by including small molecule systems such as a mixture of oxidized/reduced glutathiones and other small molecule sulfhydryl reduction/oxidation systems (e.g. dithiothreitol) to faciliate disulfide bond rearrangement. Furthermore, the addition of other molecular chaperones such as protein disulfide isomerase, cis-trans peptidyl prolyl isomerases, addition chaperone proteins such as procaryotic or eucaryotic hsp70/40/grpE like systems, small heat shock proteins, and the hsp 100 family can also augment the chaperoninlosmolyte system. Methionine sulfoxide reductase can be included in the system to insure that any inappropriately oxidized methionine residues are re-reduced after being the protein is released from the chaperonin/osmolyte system.

### EXDAMPLE 8

### Use of Method on Other Substrates and with Other Osmolytes

The chaperonin/osmolyte method will work on other protein substrates. FIG. 13 shows the method in use to refold MDH using the GroEL chaperonin, the osmolyte glycerol and ATP (shown by filled triangle). Glycerol was used in a 35% concentration.

Also shown is the effect of GroEL alone on MDH reactivation (filled squares) which can be seen to be an arresting of the refolding process. The filled diamonds show the effect of GroES to GroEL, glycerol and ATP system. Finally, the spontaneous refolding data for MDH in the presence of 35% glycerol is shown by the filled circles. Note that except for the GroEL alone, all yield measurements are within the precision of the assay measurements.

Yield of folded protein data for refolding of MDH in the presence of chaperonins or osmolytes is shown below in Table 3. These results show that MDH can be refolded with other osmolytes besides glycerol.

TABLE 3
A comparison of MDH renaturation in the presence of
GroEL/GroES ATP or with other osmolyte compounds.

| additive | percent original activity recovered* |
|---|---|
| GroEL/ES | 60 ± 13 |
| Glycerol (4M) | 60 ± 12 |
| Sucrose (1M) | 95 ± 8 |
| Betaine (1M) | 78 ± 30 |
| TMAO** (1M) | 36 ± 20 |

*At least 3 different series were measured with three replicates per series.
**TMAO—trimethylamine N-Oxide.

### V. SCREENING

The process of protein folding, in both its theoretical and practical aspects, is currently the focus of intense research. Because of the inherent complexity and variability of protein structures, it is unlikely that a single universal folding methodology, applicable to all or even a majority of the proteins, could ever be devised. One only has to note that there are multitudes of folding techniques that work only with a limited number of proteins. With the increasing amount of protein sequence information available, there is the need for a rapid and efficient screening procedure to identify the optimal protein folding solutions for specific proteins of interest.

In the present invention, a method for screening for an optimal protein folding environment for a denatured protein is provided which comprises providing a protein that needs to be folded binds to an chaperonin (e.g. GroEL), which is preferably immobilized, to form an immobilized chaperonin-protein substrate complex and then adding various osmolytes this immobilized complex. The screening systems using multiple well containing immobilized chaperonins to identify optimal osmolyte systems of single osmolyte or osmolyte mixtures. Upon identifying optimal solution conditions from screening, larger column support systems containing immobilized chaperonins are used to generate correctly folded protein with high purity, high folding yields and at high concentrations (usually greater than about 1 mg/ml quantities). Alternatively, refolded proteins can be separated from the immobilized chaperonin using ultrafiltration centrifugation technologies (Amicon ultrafiltration cells).

The chaperonin (e.g., GroEL) is used to capture and hold folding intermediates, thus preventing off path-way aggregation while forming stable long-lived (hours to days) complexes. The stable chaperonin-protein folding intermediate complex can be purified and concentrated in solution or can function while attached to an immobilized support. In the next step of the process, the chaperonin-protein folding intermediate complex can be introduced into an array of various osmolyte solutions where folding can occur directly or upon the addition of ATP or ADP (no GroES co-chaperonin required). Since the osmolyte effects on protein folding are highly variable, this provides a method to identify the superior chaperonin/osmolyte array conditions. The unique nature of this technique depends on the sequential formation of the chaperonin-protein folding intermediate complex. For example, once formed, the stable GroEL-folding intermediate complex can be concentrated to enable the testing proteins at very high concentrations (usually greater than 5 mg/ml) in small or large scales. This is significant because refolding at high concentrations is often limited due to improper mixing or competing off-pathway aggregation kinetics. Thus, it is evident that the chaperonin/osmolyte screening process possesses numerous advantages allows a high-throughput protein folding array.

Again, the GroEL capture system provides an exemplary model for the folding array. Because promiscuous GroEL hydrophobic binding site non-specifically binds a wide range of general hydrophobic folding intermediates, the high affinity GroEL species, generated by removing any bound nucleotide, can accommodate and hold an extremely large number of different protein substrates. Not only can GroEL bind a large array of different folding substrates, it can also stabilize these substrates against aggregation and the folding substrates remain bound to the chaperonin in a foldable form for a relatively long period of time. The high affinity nucleotide-free GroEL is an efficient and stable capture system for folding intermediates, preventing or arresting o.-pathway aggregation by sequestering transient kinetic folding intermediates. In some ways, the chaperonin can be compared to a non-specific antibody that binds folding intermediates typically with subnanomolar binding affinities. Once the intermediate is captured, the folding substrate is easily released from GroEL in a controlled manner.

GroEL is produced in abundance and can be purifed in 1 g quantities making it a reasonable biological tool to construct easy to use chaperonin/osmolyte folding arrays. Furthermore, a cold acetone precipitation/purification scheme removes potential interfering background peptide populations from GroEL. See Voziyan, P. A. and Fisher, M. T. (2000) Protein Sci. 9, 2405-2412, which is incorporated by reference. This protocol was used to purify three isoforms of the chaperonin from Rhizobium. See George et al., 2004, Biochem. Biophys. Res Comm. 324, 822-828, which is incorporated by reference. This purification technique is also used to functionally regenerate immobilized GroEL.

### EXAMPLE 9

### Addition of Osmolytes to Different Test Substrate Proteins

In this example, the screening method of the present invention identified proteins which fail to correctly fold with the complete GroE chaperonin system (GroEL, GroES ATP) or with osmolytes alone can correctly fold when GroEL and osmolytes are combined. In addition, it was found that some commonly used osmolytes will facilitate the renaturation of stringent chaperonin substrates without requiring GroES. Stringent chaperonin substrates are generally defined as those proteins that absolutely require the complete GroE chaperonin system (GroEL, GroES and ATP) to fold.

In the foregoing examples, it was shown that the GroEL/osmolyte system could successfully fold a GS truncation mutant and a stringent chaperonin substrate (MDH). This example expanded the substrate protein test set to examine the broader folding efficiency of the GroEL/osmolyte system. This example includes other substrate proteins that are difficult to fold, as well as two proteins that were isolated and purified from inclusion bodies. In all but one case, these test proteins were able to be efficiently with folded GroEL, nucleotide and an osmolyte. Firefly luciferase was the only protein that could not fold with GroEL, nucleotide and osmolytes alone and required the presence of GroES. From the data shown in Table 4, it is clear that many different osmolytes facilitate folding from GroEL and ATP or ADP. In some instances, folding could be accomplished by just adding osmolytes to GroEL without adding any nucleotide. Furthermore, for all folding substrates tested, the stringent requirement of ATP is also relaxed because it was found that ADP can be used to initiate successful folding from the chaperonin in the presence of select osmolytes (Table 4). Thus, osmolytes simplify the chaperonin reaction because they may eliminate the requirement to add GroES (70 kDa) (another potential small protein contaminant) and they may eliminate the need to add and maintain ATP levels to sustain the refolding reaction. The induction of successful protein refolding in the presence of GroEL, osmolyte and ADP makes this assay easier to control and run.

For this example, GroEL was prepared as described previously. See 1. Voziyan, P. A. and Fisher, M. T. (2000) Protein Sci. 9, 2405-2412. Porcine citrate synthase, mitochondrial and cytoplasmic porcine malate dehydrogenases (MDHs), horse liver alcohol dehydrogenase, and rhodanese were purchased from Sigma. Firefly luciferase was purchased from Promega. Glutamine synthetase (GS) purification from E. coli and activity measurements were performed as previously described by Fisher and Stadtman, (1992) J. Biol. Chem. 267,1872-1880. Cell extracts containing phosphoinositol transfer protein (PITP) aggregates were provided by G. Helmkamp. Inclusion bodies were prepared and purified according to the procedures described by Georgiou, G. and Valax, P. (1999) Methods Enzymol. 309, 48-58 Matrix protein inclusion bodies (unidentified) were purified and soluble protein was assessed and detected using Western blot. The chaperonin-protein folding intermediates were prepared as previously described in Voziyan, P. A., Jadhav, L. and Fisher, M. T. (2000) J. Pharm. Sci. 89, 1036-1045 and Voziyan, P. A. and Fisher, M. T. (2002) Arch. Biochem. Biophys. 397, 293-297.

TABLE 4
Test set of substrate proteins used for refolding with the GroEL/osmolyte system.

| | Refolding | | | |
|---|---|---|---|---|
| | alone; no | | GroEL + best | GroEL + ADP |
| | GroEL or | Best osmolyte | osmolyte | or ATP + best |
| Substrate protein | smolyte | or additives | (no ATP or ADP) | osmolyte |
| Glutamine synthetase | 10% | Glycerol, | 60-80% | 60-80% |
| | | sucrose, or | (Glycerol) | |
| | | trimethylamine | | |
| | | N-oxide | | |
| rhodanese | ˜3-5% | Glycerol | No release and | 45-60% |
| | | | refolding from | |
| | | | GroEL^{a} | |
| Mitochondrial malate | ˜5% | Sucrose | No release and | 70-80% |
| dehydrogenase (MDH) | | | refolding from | |
| | | | GroEL^{a} | |
| Cytoplasmic DH | ˜60% | Glycerol | ˜60-75% | 70-80% |
| | | | (Glycerol) | |
| Citrate synthase | ˜2-5% | Glycerol | No release and | 45-60% |
| | | | refolding from | |
| | | | GroEL^{a} | |
| Horse liver alcohol | 41% | Glycerol or | 45-50% | 45-50% |
| dehydrogenase | | sucrose | (Glycerol) | |
| Firefly luciferase | 5-6% | None-(requires | >5% | >5% |
| | | complete GroE | | |
| | | system) 60-69%) | | |
| Phosphoinositol | 3% | L-proline or | ND^{c} | 70-80% |
| transfer protein (PITP)^{b} | | sarcosine | | |
| Matrix protein^{b} | No soluble | L-Arginine | No release | Soluble protein |
| | protein | | | |

^{a}No release from GroEL alone was observed until ATP (or ADP) was added (next column).
^{b}Refolded from purified inclusion bodies.
^{c}Not determined.

### EXAMPLE 10

### Addition of Osmolytes to Immobilized Chaperonin

In this example, we showed that the addition of the test osmolytes work with immobilized GroEL (tetradecamer)-beads. This example shows that immobilized GroEL binding platforms are also capable of folding proteins in the presence of osmolytes in the same way that was observed in solution. The non-specific covalent immobilization of GroEL was easily accomplished using standard N-Hydroxysuccinimide (NHS)-activated Sepharose 4 fast flow beads (Pharmacia-Biotech). This rapid and easy coupling method results from the formation of a covalent linkage between forming very stable amide covalent linkage between the flow bead and primary amines (primarily lysines on GroEL at near neutral pH). It was found that as much as about 10 mg GroEL could be immobilized onto 0.5 ml of wet beads.

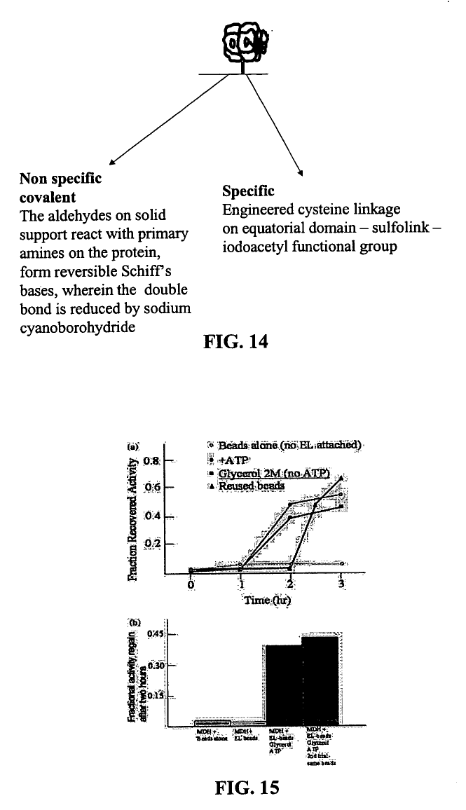

It will be appreciated, however, that a wide array of immobilization techniques can be used, such as those illustrated in FIG. 14. These include other non-specific covalent linkages. For example, the AminoLink® coupling system commercially available from Pierce Chemical can be used to produce a non-specific covalent linkage. The coupling system has aldehyde fimctional groups on a solid support which react spontaneously with primary amines on the protein (e.g. lysine residues). Reductive animation of the resulting Schiff base forms a stable secondary amine linkage. The double bond can be reduced by sodium cyanoborohydride or other suitable agents. In addition, specific covalent linkages for immobilization of the chaperonin include a sulfur linkages. For example, the SulfoLink® coupling system commercially available from Pierce Chemical includes iodoacetyl fimctional group which is covalently linked to a resident or genetically engineered thiol (e.g., site-directed mutagenesis replacement of surface residue with cysteine) linkage on the solvent accessible surface of the protein equatorial domain to form a S-carboxymethyl linkage.

The immobilized GroEL binds partially folded or unfolded substrate proteins, completely arrests any refolding and can be reused (FIG. 15a, b). For this experiment, a low concentration of folding protein (MDH or GS) was captured on the beads, and excess ATP and osmolyte was added. The beads were pelleted the beads and the supernatant was assayed for enzyme activity. The results were identical to those observed in solution.

In addition, the immobilized double ring chaperonin can be reused and is able to fold repeated additions of unfolded substrate protein. Specifically, the GroEL beads were treated with ATP and 1 M urea, washed with refolding bu.er and another sample of unfolded GS or MDH monomers (about 0.5 1 M) was captured by the bead immobilized GroEL. For the regeneration experiments, ATP and glycerol was added to the samples to reactivate dodecameric GS (FIG. 14a) or dimeric MDH (FIG. 14b). The experiments that examined the recycling ability of the immobilized chaperonins were performed the following day, indicating that the immobilized system remained active for at least one day.

### EXAMPLE 11

### Screening Systems

By demonstrating that the chaperoning, such as GroELm can be immobilized in a functional state (FIG. 14) which can be reused after multiple rounds of adding osmolytes as folding additives, this example shows that higher throughput screening systems can be constructed to test the folding success of a wide variety of protein substrates or even protein complexes from the chaperonin can be assessed after addition of osmolyte systems along with nucleotides such as ADP or ATP.

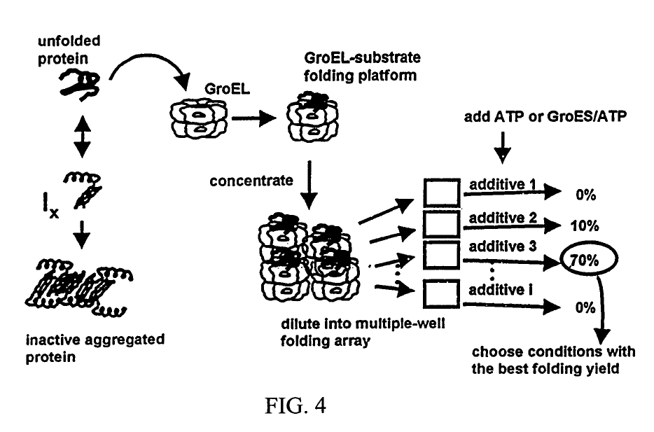

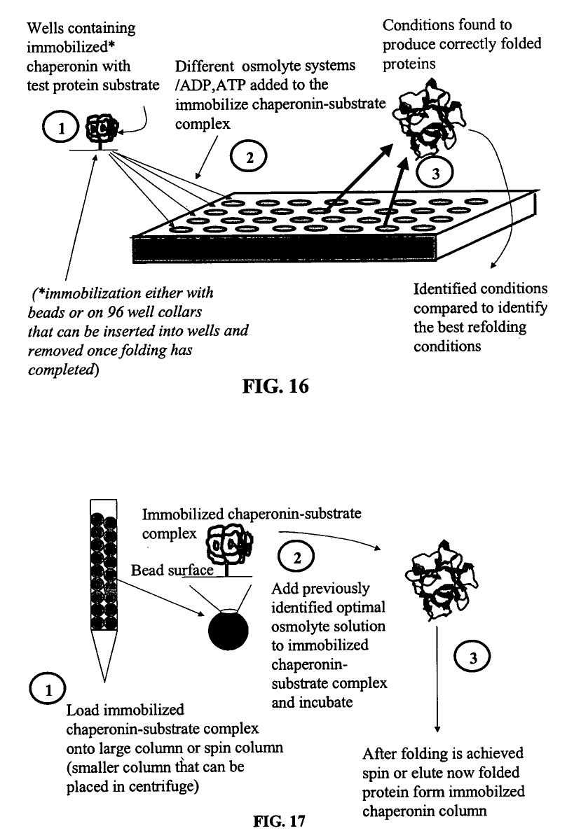

To achieve high throughput screening to identify optimal osmolyte systems, the chaperonin can be attached in multiple well arrangements (directly to the wells or to multiple well collar inserts) where the ability of the added osmolyte system within each individual well will enable one to access the folding yields under each condition (FIGS. 4 and 16) (Voziyan et al., 2000).

As an example, FIG. 4 shows that the chaperonin/osmolyte approach offers a methodology for easy testing of a wide range of folding conditions to aid in refolding of problematic proteins. The procedure starts with the formation of GroEL-protein substrate complexes, thereby preventing non-productive aggregation. Without ATP, these complexes are very stable and can be easily concentrated with virtually no loss of the protein substrate (Fisher, M. T. (1993) J. Biol. Chem. 268, 13777-13779; Smith, K. E. and Fisher, M. T. (1995) J. Biol. Chem. 270, 21517-21523). The concentrated GroEL-protein substrate complexes are then used as a platform to test a multiple array of osmolyte solutions (“folding array”) in order to identify optimal folding conditions for the protein of interest.

As each element of the folding array contains a different osmolyte solution, introducing a portion of the complex into each element of the array will test the efficacy of each osmolyte. Mutant GSΔ468 is a convenient model for the testing of the in vitro refolding procedure. Because this mutant folds to an active form in the cell, neither its folding nor its enzymatic activity have been perman&ntly disrupted by truncation. However, the refolding of this protein in vitro represents a considerable challenge since it does not refold either spontaneously or with the major bacterial molecular chaperone systems.

There are a number of advantages to the present screening invention. The first unique aspect of this invention is the demonstration that the chaperonin can capture the folding intermediate, arresting further deleterious misfolding and aggregation. This complex is stable (high binding affinity of a K_{d} at about 0.5 μM or below) and is the primary reason why off pathway protein misfolding reactions (i.e. aggregation) are prevented. The chaperonin can bind a wide range of partially folded, misfolded or even completely unfolded protein folding intermediates. By this fact alone, the range of proteins that the chaperonin can potentially fold is much greater than more commonly used commercially available folding screens.

Another aspect involves the user ability to change the solution folding environment of the chaperonin captured protein by adding the osmolyte system of choice. Once bound to the chaperonin, various osmolyte systems that are added to the chaperonin-protein folding intermediate complex have been shown to facilitate protein folding. These numerous osmolyte systems have varying positive effects on the bound intermediate and as a result, the outcome or success of each osmolyte system can only be determined using a screening method. Since the folding intermediate starts from the same state (chaperonin bound), the screening starts from the same starting point no matter which osmolyte system is used. Conversely, other common folding screens have to rely and hope that the particular additive solutions alone prevent misfolding and aggregation of a free folding intermediate. Furthermore, it is observed the osmolyte or other solution additives alone will cause the protein to aggregate and misfold (Voziyan and Fisher, 2000). The sequential nature of first forming the chaperonin substrate complex provides a very unique advantage over the other common folding screens because the chaperonin bound intermediates all start from the same state prior to osmolyte system addition.

The next important aspect of this invention relies on the fact that the chaperonin-protein folding complex can be concentrated without the loss of protein product due to aggregation. Current refolding protocols outlined within current in vitro folding kits are not able to extend this folding concentration range as easily as can be accomplished with the chaperonin/osmolyte protein folding system.

The last advantage of this invention, particularly for the development of high throughput screens to optimize folding and folding/purification, involves the ability to be able to immobilize the chaperonin using a wide array of chemically available immobilization reactions. In every example, the folding ability of the chaperonin in solution is recapitulated if one uses an immobilized version of the chaperonin. Thus, the immobilized chaperonin can be easily removed from the folding solution, allowing the protein to continue to fold without rebinding to the chaperonin, and allowing the experimenter to reuse the attached chaperonin for another round of protein substrate capture and release.

### EXAMPLE 12

### Large Capacity Folding Procedures

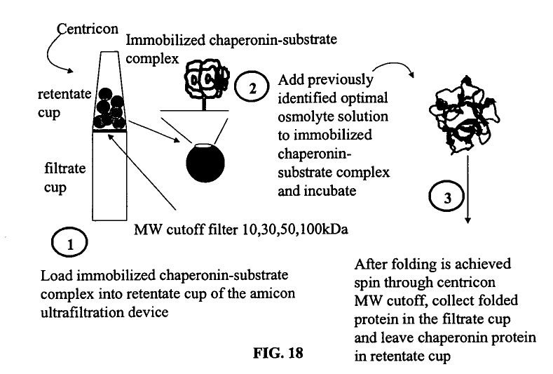

Once the optimal osmolyte solution has been identified by this small scale high-throughput screening process, the larger capacity folding procedure can then be implemented. In this procedure, the protein to be folded is first bound in bulk to an immobilized chaperonin construct attached to commercially available immobilization beads and placed either into a column (FIG. 17) for column chromatography or into an Amicon® centricon (FIG. 18). The optimal osmolyte solution(s) identified by the high throughput screen is then added to the column or centricon immobilized chaperonin-protein substrate complex along with ADP or ATP, the protein is allowed to dissociate and fold and the folded product is removed and collected in the flow through (in the case of column) or in the ultrafiltration technology, separated by molecular mass into the filtrate cup (FIG. 18).

More specifically, as shown in FIG. 17, the chaperonin is immobilized on a support, such as a bead, which is placed into a column. The osmolyte system previously identified as being optimal is then introduced into the column. After a sufficient time for folding to occur, the folded protein is removed from the bead immobilized chaperonin-protein complex by gravity or flow elution with the optimal osmolyte system or through centrifugation of a spin column (about 1×1500 g for one minute for larger columns). As stated previously, the refolded protein remains in the optimal osmolyte solution during the collection phase of the spin column procedure.

As another example, as shown in FIG. 18, the chaperonin is immobilized on a support, such as a bead, which is placed into ultrafiltration device. Ultrafiltration techniques generally rely on the use of polymeric membranes with highly defined pore sizes to separate molecules according to size. The technique relies on the use of centrifugation to drive the migration of the smaller folded protein molecules through the membrane to the filtrate cup with the simultaneous retention of larger molecules in the retentate cup. More specifically, after the osmolyte system (along with the nucleotides ATP or ADP) previously identified as being optimal is introduced into the device for a sufficient period of time for the protein to be released from the chaperonin and fold, the device is centrifuged according to manufacturer specifications so that the the folded protein is collected in the filtrate cup, while the immobilized (or even soluble) chaperonin protein remains in the retentate cup. See generally U.S. Pat. No. 6,357,601 entitled “Ultrafiltration device and method of forming same” and U.S. Pat. No. 4,755,301 entitled “Apparatus and method for centrifugal recovery of retentate.”

In sum, in the present invention it was shown that although both GroE chaperonins and cellular osmolytes have been used before individually to enhance protein folding, a combination of these methods in the two-step folding procedure provides several important and unexpected benefits. The procedure combines the chaperonin's ability to prevent aggregation and even unfold the misfolded intermediates with the inherent structural stabilization and enhancement of folding afforded through the use of osmolytes. As the experiments with GSΔ468 demonstrate in Table 1, this combination can produce a remarkable synergistic amplification of protein folding in vitro. Because the refolding of denatured protein is performed in two steps, the solution parameters such as temperature, ionic strength, and protein concentration can be adjusted independently to insure both the efficient chaperonin-substrate complex formation and the optimal substrate release and refolding in the presence of osmolytes. The high stability of the complex allows for an easy manipulation of solution conditions without the significant loss of the folding proteins due to aberrant aggregation at higher concentrations. In the case of GSΔ468, substrate concentration was initially kept low in order to avoid rapid aggregate formation and insure high chaperonin-to-substrate stoichiometry. Once the complex is formed, however, the substrate concentration can be increased to enhance the concentration-dependent second order GSΔ468 assembly reaction as shown in Table 2.

Because GroEL interacts mainly with the exposed hydrophobic surfaces of folding intermediates, it is capable of binding of a wide variety of proteins without apparent specificity (for review, see Fenton, W. A. and Horwich, A. L. (1997) Protein Sci. 6, 743-760). The stabilizing effect of osmolytes has been shown for a number of structurally diverse proteins and, in general, is related to the change in hydration of the macromolecular surface (Wang, A. and Bolen, D. W. (1997) Biochemistry 36, 9101-9108; De-Sanctis, G., Maranesi, A., Ferri, T., Poscia, A., Ascoli, F., and Santucci, R. (1996) J. Protein. Chem. 15, 599-606; Chen, B. L. and Arakawa, T. (1996) J. Pharm. Sci. 85, 419-426; Zhi, W., Landry, S. J., Gierasch, L. M., and Srere, P. A. (1992) Protein Science 1, 552-529). These general mechanisms of action of chaperonins and osmolytes suggest that the proposed folding method may be applicable to a relatively wide variety of proteins, regardless of their specific structural features. Indeed, besides GSΔ468, osmolyte-induced decrease in chaperonin requirements (i.e., when GroES and, in some cases, ATP were no longer required) for refolding of mitochondrial malate dehydrogenase, bovine rhodanese, and wild-type GS have been observed.

In the present invention, it was also shown that the formation of stable chaperonin-substrate complexes, the two-step refolding procedure, and a multiple-well “folding array” allow one to screen a broad range of folding solution conditions for a particular protein of interest. Unlike other screening protocols (Chen, G-Q. and Gouaux, E. (1997) Proc. Natl. Acad. Sci. USA 94, 13431-13436, the disclosure of which is incorporated herein by reference), methods of the present invention ensures that initial aggregation of now stable protein folding intermediate does not occur. For the screening, protein folding efficiency could be monitored either by measuring protein enzymatic activity or by following spectroscopic or other structurally sensitive parameters that characterize protein conformation. In an earlier study, the matrix-immobilized GroEL-GS and GroEL-tubulin complexes were used to refold corresponding proteins (Phadtare, S., Fisher, M. T., Yarbrough, L. R. (1994) Biochim. Biophys. Acta. 1208, 189-192, the disclosure of which is incorporated herein by reference). In these cases, however, problems with protein release and aggregation limited the broad applicability of the technique (Phadtare, S., Fisher, M. T., Yarbrough, L. R. (1994) Biochim. Biophys. Acta. 1208, 189-192). Coupling of this technique with the chaperonin/osmolyte folding array approach potentially allows one to obtain preparative quantities of the protein of interest using column chromatography. In another solid support-based approach the attachment of protein substrate to the matrix was achieved using the monomeric fragments of GroEL apical domains (Altamirano, M. M., Golbik, R., Zahn, R., Buckle, A. M., and Fersht, A. R. (1997) Proc. Natl. Acad. Sci. USA 94, 3576-3578; Altamirano, M. M., Garcia, C., Possani, L. D., and Fersht, A. R. (1999) Nat. Biotechnol. 17, 187-191). Although these “mini-chaperones” can enhance protein refolding in some cases (Zahn, R., Buckle, A. M., Perrett, S., Johnson, C. M., Corrales, F. J., Golbik, R., and Fersht, A. R. (1996) Proc. Natl. Acad. Sci. USA 93, 15024-15029, the disclosure of which is incorporated herein by reference), they completely fail to arrest protein folding and cannot substitute for oligomeric GroEL in the enhancement of folding (Weber, F., Keppe, F., Georgopoulos, C., Hayer-Hartl, M. K., and Hartl, F. U. (1998) Nat. Struct. Biol. 5, 977-985, the disclosure of which is incorporated herein by reference). It appears, therefore, that the use of the oligomeric GroEL chaperonin is better suited for capturing, stabilizing, and immobilizing aggregation-prone protein substrates on a matrix where optimal solution conditions for successful release and refolding can be tested in a broad manner. As this invention with GSΔ468 demonstrates, at certain solution conditions GroES can be completely removed from the folding protocol without compromising folding yields, an important consideration when a large-scale refolding and purification procedures have to be performed.

Although the model protein GSΔ468 folded successfully in cellular environment, it failed to refold with bacterial GroE and DnaK chaperone systems in vitro. These data imply that cytosol components other than the above molecular chaperones could be essential for productive folding of mutant GS. It is certainly possible that the low molecular weight solutes within the bacterial cytoplasm may play a significant role in facilitating protein folding. Indeed, one of the compounds that enhanced chaperonin-dependent GSΔ468 refolding in our experiments was 0.5 M potassium glutamate. These conditions are particularly interesting because the physiological concentration of potassium and glutamate ions in E. coli cells has been shown to be in a range of 0.2-1 M (Richey, B., Cayley, D. S., Mossing, M. C., Kolka, C., Anderson, C. F., Farrar, T. C., and Record, M. T., Jr. (1987) J. Biol. Chem., 262, 7157-7164, the disclosure of which is incorporated herein by reference). It is possible that the other natural osmolytes found in many bacterial, plant, and mammalian cells (Sola-Penna, M., Ferreira-Pereira, A., Lemos, A. P., and Meyer-Femandes, J. R. (1997) Eur. J. Biochem. 248, 24-29; Yoshiba, Y; Kiyosue, T; Nakashima, K; Yamaguchi-Shinozaki, K; Shinozaki, K (1997) Plant. Cell. Physiol. 38, 1095-10102; Paredes, A; McManus, M; Kwon, H M; Strange, K. (1992) Am. J. Physiol. 263, C1282-1288; Warskulat, U; Wettstein, M; Haussinger, D (1997) Biochem. J. 321, 683-690; Record, M. T., Jr., Courtenay, E. S., Cayley, S., and Guttman, H. J. (1998) Trends Biochem. Sci. 23, 190-194, the disclosures of which are incorporated herein by reference), in conjunction with molecular chaperones, could also enhance the intracellular protein folding kinetics and stability, and may represent a more complete system that describes protein folding mechanism in the cell. For example, TMAO, a natural osmolyte found in a number of marine species (Yancey, P. H., Clark, M. E., Hand, S. C., Bowlus, R. D., and Somero, G. N. (1982) Science 217, 1214-1222), facilitates the refolding of GSΔ468 in the presence of chaperonins.

The evolutionary selected cellular solution conditions arguably represent the best system for folding the intrinsic proteins. The present invention demonstrates that a combination of two natural cellular components, chaperonins and osmolytes, can dramatically improve folding yields for a protein whose in vitro folding reaction is problematic.

While the present invention has been described herein with reference to the particular embodiments thereof, a latitude of modifications, various changes and substitutions are intended in the foregoing disclosure, and it will be appreciated that some features of the invention will be employed without a corresponding use of other features, without departing from the scope of the invention as set forth.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

1. A method of screening for an optimal folding environment for a denatured polypeptide, comprising the steps of:
(a) providing a polypeptide in an unfolded state which is capable of binding to a chaperonin;
(b) binding said polypeptide to said chaperonin to form chaperonin-polypeptide complexes for the folding of said polypeptide to its active state;
(c) providing a folding array having a plurality of elements with each element having comprising a different osmolyte therein;
(d) introducing a portion of said complexes to each of said elements in said array thereby adding each of said different osmolytes to said chaperonin-polypeptide complex thereby promoting, to varying degrees, the folding of said polypeptide from its unfolded state to its folded state to yield a folded, biologically active polypeptide; and
(e) identifying the most efficient folding conditions for said polypeptide by measuring the yield of folded polypeptides within each element of said array.

2. The method of screening of claim 1 wherein said chaperonin is of the Escherichia coli GroE chaperonin family.

3. The method of screening of claim 2 in which the chaperonin is E. coli GroEL.

4. The method of screening of claim 1 in which one of said different osmolytes is sucrose.

5. The method of screening of claim 1 in which one of said different osmolytes is glycerol.

6. The method of screening of claim 1 in which one of said different osmolytes is trimethylamine N-oxide.

7. The method of screening of claim 1 in which one of said different osmolytes is potassium glutamate.

8. The method of screening of claim 1 in which one of said different osmolytes is arginine.

9. The method of screening of claim 1 in which one of said different osmolytes is betaine.

10. The method of screening of claim 1 in which one of said different osmolytes is urea.

11. The method of screening of claim 1 in which one of said different osmolytes is sarcosine.

12. The method of screening of claim 1 in which one of said different osmolytes is L-proline.

13. The method of screening of claim 1 further comprising the step of promoting the folding of said polypeptide by the addition of a co-chaperonin to the chaperonin-polypeptide complex, wherein said co-chaperonin has the ability to bind and dissociate from the chaperonin and aid said chaperonin to achieve correct binding of said polypeptide.

14. The method of screening of claim 1 wherein said chaperonin is immobilized on an inert support.

15. The method of screening of claim 1 where in the concentration of said osmolyte is sufficient to substantially prevent the aggregation of the unfolded polypeptides into unusable forms.

16. The method of screening of claim 1 wherein said unfolded polypeptide is incapable of being folded to its biologically active form by either a chaperonin or an osmolyte alone.

17. The method of screening of claim 1 wherein said method is conducted under controlled oxidation/reduction conditions.

18. The method of screening of claim 17 in which the oxidation/reduction conditions comprise an at least substantially anaerobic envirornent.

19. The method of screening of claim 17 wherein said oxidation/reduction conditions are controlled by one or more redox agents selected from the group consisting of glutathione, sulfhydryl and protein reduction systems.

20. The method of screening of claim 1 wherein said identifying step comprises monitoring protein enzymatic activity.

21. The method of screening of claim 1 further comprising the step of adding a nucleotide to the chaperonin-polypeptide complex with the addition of the osmolyte.

22. The method of screening of claim 21 wherein said nucleotide is selected from the group consisting of ATP or ADP.

23. A folding array for selecting optimal folding environment for a denatured polypeptide, comprising:
a chaperonin immobilized on a support; and
a plurality of elements each element having comprising a different osmolyte therein.

24. The folding array of claim 23 wherein said support is a bead.

25. The folding array of claim 23 wherein said chaperonin is immobilized non-specifically to said support using a covalent amino linkage through a chaperonin residue containing a primary amine on the chaperonin.

26. The folding array of claim 23 wherein said chaperonin is immobilized specifically to said support using a covalent sulfo-linkage through an chaperonin cysteine residue.

27. The folding array of claim 23 wherein said plurality of elements comprises a multiple-well array.

28. A method for purification and isolation of a folded protein comprising:
(a) providing a polypeptide in an unfolded state which is capable of binding to a chaperonin;
(b) binding said polypeptide to said chaperonin to form chaperonin-polypeptide complexes for the folding of said polypeptide to its active state;
(c) providing a folding array having a plurality of elements with each element having comprising a different osmolyte therein;
(d) adding an osmolyte to said chaperonin-polypeptide complex in order to promote the folding of said polypepide to its folded state, said osmolyte system being identified using the method of screening of claim 1;
(e) removing said polypeptide from said chaperonin-polypepide complex to yield an isolated folded polypeptide.

29. The method for purification and isolation of a folded protein of claim 28 where said chaperonin is immobilized on a support, and said removing step is performed by ultrafiltration.

30. The method for purification and isolation of a folded protein of claim 28 where said chaperonin is immobilized on a support, and said removing step is performed by column chromatography.
